(19) United States
(12) Patent Application Publication
(10) Pub. No.: US 2006/0252992 A1
(43) Pub. Date: Nov. 9, 2006

(54) Flexible tube for endoscope

(75) Inventor: Naotake Mitsumori, Saitama-shi (JP)

Correspondence Address: BIRCH STEWART KOLASCH & BIRCH, PO BOX 747, FALLS CHURCH, VA, 22040-0747, US

(73) Assignee: Fujinon Corporation

(21) Appl. No.: 11/392,835

(22) Filed: Mar. 30, 2006

(30) Foreign Application Priority Data
Mar. 31, 2005 (JP) P.2005-102264
Apr. 28, 2005 (JP) P.2005-132859
May 24, 2005 (JP) P.2005-151129

(51) Int. Cl. A61B 1/00

(52) U.S. Cl. 600/139

(57) ABSTRACT

A flexible tube which is for an endoscope and has flexibility across almost an entire length in its bending direction, the flexible tube comprises: a tubular structure with flexibility in its bending direction; a tubular mesh that covers the tubular structure; and an outer sheath layer comprising a urethane resin, the outer sheath layer being laminated on the tubular mesh, wherein a tube layer formed from a chemical resistant rubber material is provided between the tubular structure and the tubular mesh.

### BACKGROUND OF THE INVENTION

1. Field of the Invention

The present invention relates to a flexible tube for an endoscope, and in particular, to a flexible tube for an endoscope excellent in chemical resistance and durability. The present invention relates to a structure of a flexible tube for an endoscope, and in particular, to a flexible tube for an endoscope whose degree of flexibility in a bending direction changes in the axial direction.

2. Description of the Related Art

In an endoscope to be widely used for medical use, as shown in FIG. 5, a base end of an insertion portion 101 to be inserted into a body cavity is coupled to a main body control portion 102, and a light guide flexible portion 103 extends from this main body control portion 102. In the insertion portion 101, the greater part of the length from a coupling side to the main body control portion 102 is a flexible tube portion 101a, and at the distal end of this flexible tube portion 101a, an angle portion 101b that can be bent upward, downward, leftward, and rightward by an angle control means 104 provided at the main body control portion 102 is provided, and a distal end portion main body 101c is coupled to the distal end of this angle portion 101b.

A flexible tube 100 forming the flexible tube portion 101a is constructed so that, as shown in FIG. 6, a spiral tube 11 formed by spirally winding a metal band at the innermost side is covered by a tubular mesh 12 formed by weaving a metal wire, and an outer sheath layer 13 made of an urethane resin or the like is laminated on this tubular mesh 12. Thereby, a flexible tube portion 101a having flexibility in its bending direction and sufficient strength in its expanding and contracting direction and crushing direction can be obtained.

Endoscopes for medical use are required to be completely disinfected and sterilized, and in particular, the insertion portion 101 to be inserted into a body cavity is required to be highly disinfected and sterilized. The urethane resin forming the outer sheath layer 13 of the flexible tube 100 is low in resistance against a chemical to be used for disinfection and sterilization (for example, hydrogen peroxide plasma, peracetic acid, (CH₃COOH), or acid water, etc.), so that to prevent the outer sheath layer from being broken (corroded) by the chemical, the outer surface of the outer sheath layer made of a urethane resin is covered by a coating film containing, for example, silicon or the like having chemical resistance.

However, recently, gas sterilizers that sterilize endoscopes by using a sterilizing gas such as hydrogen peroxide plasma have spread, and if an endoscope is sterilized with the gas sterilizer, even when the coating film with chemical resistance is covered on the outer surface of the outer sheath layer made of a urethane resin, the sterilizing gas entering inside the endoscope breaks the outer sheath layer made of a urethane resin from the inner side.

When an endoscope is sterilized with the gas sterilizer, to prevent the endoscope from being broken due to an air pressure difference between the inside and the outside of the endoscope, a leak valve that communicates the inside air and the outside air of the endoscope with each other is opened to perform disinfection and sterilization (gas sterilization) by using the sterilizing gas. Then, when the gas sterilization is performed by opening the leak valve, the sterilizing gas enters the inside of the endoscope and breaks the urethane resin forming the outer sheath layer of the flexible tube for the endoscope from the inner side.

Therefore, development of a flexible tube for an endoscope which prevents the outer sheath layer from being broken by a sterilizing gas entering inside the endoscope has been demanded (for example, refer to JP-A-2002-95628).

For example, as a flexible tube for an endoscope that prevents an outer sheath layer from being broken by a sterilizing gas entering inside the endoscope, it is considered that the urethane resin as a material forming the outer sheath layer is changed to a chemical resistant resin such as a fluorine resin.

However, the endoscope requires sensitive control, and if the outer sheath layer is made of a material other than the urethane resin, the control feeling of the endoscope (bending easiness and a certain level of elasticity) changes, so that such material change that provides a doctor who handles the flexible tube portion with a sense of discomfort is undesirable.

In addition, in a flexible tube provided with an outer sheath layer containing a fluorine resin at the inner side of the urethane resin as shown in JP-A-2002-95628, adhesiveness between the fluorine resin and the tubular mesh is low, so that this requires a means to increase the adhesiveness between the outer sheath layer and the tubular mesh to prevent separation between the outer sheath layer and the tubular mesh. The material disclosed in JP-A-2002-95628 is insufficient in chemical resistance. (The above is referred to as a first problem.)

The flexible tube portion 101a needs to have flexibility across almost the entire length in its bending direction, and the side continued to the main body control portion 102 (hereinafter, referred to as a base end side) needs considerably high rigidity against bending in order to make excellent a pushing thrust for inserting into a body cavity. On the other hand, desirably, the side continued to the angle portion 101b (hereinafter, referred to as an angle side) has a higher degree of flexibility so as to follow the bend of the angle portion 101b to some degree and smoothly bends along the curved insertion path. Therefore, it is advantageous in terms of inserting operability and pain reduction for a patient that the degree of flexibility of the flexible tube portion 101a is changed in the axial direction, that is, the base end side is hard and the angle side is flexible.

Therefore, in the flexible tube 100 for an endoscope according to the related-art technique, as shown in FIG. 9, an outer sheath layer 13 formed by combining a high-hardness resin layer 13a made of a hard resin material and a low-hardness resin layer 13b made of a soft resin material is laminated, whereby a flexible portion and a hard portion are formed in the flexible tube 100.

In addition, the degree of flexibility in the bending direction changes in the axial direction of the flexible tube 101a for an endoscope, the base end side of the flexible tube portion 101a is made hard to form a high-hardness flexible portion, and the angle side is made flexible to form a low-hardness flexible portion (for example, refer to JP-A-2001-238851 and JP-A-63-249536).

However, in the structure in which the degree of flexibility in the bending direction is changed in the axial direction of the flexible tube portion 101a by laminating the outer sheath layer 13 made of a combination of different resin materials (hard resin material and soft resin material), repeated bending stresses of the flexible tube portion 101a cause cracks and the like at the interface between the resin materials. (The above is referred to as a second problem.)

### SUMMARY OF THE INVENTION

To solve the first problem, according to the invention, there is provided a flexible tube which is for an endoscope and has flexibility across almost an entire length in its bending direction, the flexible tube comprising: a tubular structure with flexibility in its bending direction; a tubular mesh that covers the tubular structure; and an outer sheath layer comprising a urethane resin, the outer sheath layer being laminated on the tubular mesh, wherein a tube layer formed from a chemical resistant rubber material is provided between the tubular structure and the tubular mesh.

In addition, a tube layer formed from a rubber material having a perfluoro monomer structure is provided further inward than the outer sheath layer comprising a urethane resin.

According to the flexible tube for an endoscope of the invention, by providing a tube layer made of a chemical resistant rubber material between the tubular structure and the tubular mesh, chemical resistance can be secured in the outer sheath layer without changing the urethane resin forming the outer sheath layer, and this eliminates the possibility that a doctor handling the endoscope is provided with a sense of discomfort in control feeling when inserting the insertion portion into a body cavity of a patient. In addition, by forming convex portions on the inner side of the tube layer covered on the outer circumference of a spiral tube (tubular structure) formed by spirally winding a metal band and interposing the convex portions in between-band portions of the spiral tube at arbitrary pitches corresponding to the spiral intervals, the flexibility (hardness) of the flexible tube can be easily adjusted.

In a flexible tube further provided with a tube layer made of a rubber material having a perfluoro monomer structure further inward than the outer sheath layer, excellent chemical resistance is obtained and adaptation to autoclaving sterilization (a method of sterilization by using water steam heated and pressurized to 2 atmospheres and 132° C.) is also possible.

To solve the second problem, according to the invention, there is provided a flexible tube which is for an endoscope and has flexibility across almost an entire length in its bending direction, the flexible tube comprising: a tubular structure with flexibility in its bending direction; a tubular mesh that covers the tubular structure; and an outer sheath layer laminated on this tubular mesh, wherein the outer sheath layer is formed by crosslinking a vulcanizing-type rubber material having a perfluoro monomer structure, and a molecular weight of the rubber material is arbitrarily changed in a range between 1000 and 2000 along an axial direction of the outer sheath layer, so that degree of flexibility in the bending direction is changed in the axial direction.

According to the flexible tube for an endoscope of the invention, when a vulcanizing-type rubber material having a perfluoro monomer structure is crosslinked (vulcanized) to form the outer sheath layer, the molecular weight of the rubber material is arbitrarily changed along the axial direction, whereby the flexibility of the outer sheath layer can be easily changed in the axial direction.

Namely, by controlling the crosslinking reaction, without combining different resin materials, the degree of flexibility in the bending direction can be changed in one material in the axial direction of the flexible tube for an endoscope, and a flexible tube portion whose base end side is formed into a high-hardness flexible portion with low flexibility and angle side is formed into a low-hardness flexible portion with high flexibility can be easily formed.

In addition, the flexible tube made of a vulcanizing-type rubber material having a perfluoro monomer structure with a molecular weight of 2000 or less has excellent chemical resistance and is adaptable to autoclaving sterilization (sterilization by using water vapor heated and pressurized to 2 atmospheres and 132° C.), and is small in friction resistance and excellent in smoothness, and has no harmful influence on human bodies.

To solve the second problem, according to the invention, there is provided a flexible tube which is for an endoscope and has flexibility across almost an entire length in its bending direction, the flexible tube comprising: a tubular structure with flexibility in its bending direction; a tubular mesh that covers the tubular structure; an outer sheath layer comprising a rubber material, the outer sheath layer laminated on the tubular mesh, wherein the tubular structure is a spiral tube formed by spirally winding a metal band, the spiral tube comprising between-band portions, a thin-film resin layer having convex portions formed on its inner side is provided between the tubular structure and the tubular mesh so that the convex portions of the resin layer are interposed at arbitrary pitches in between-band portions of the spiral tube, and degree of flexibility in the bending direction is changed in the axial direction by arbitrarily changing the pitches of the convex portions interposed in the between-band portions of the spiral tube along an axial direction of the spiral tube.

According to the flexible tube for an endoscope of the invention, a thin-film resin layer having convex portions formed on the inner side is provided between the tubular structure and the tubular mesh, the convex portions are interposed in between-band portions of the spiral tube at arbitrary pitches, and the degree of flexibility of the outer sheath layer is easily changed by controlling the pitches of the convex portions interposed in the between-band portions.

Namely, by changing the pitches of the convex portions of the resin layer between the tubular structure and the tubular mesh interposed in between-band portions of the spiral tube, the degree of flexibility of the flexible tube for the endoscope in the bending direction can be changed in the axial direction without providing an outer sheath layer made of a combination of different resin materials and a flexible tube portion whose base end side is formed into a high-hardness flexible portion with low flexibility and angle side is formed into a low-hardness flexible portion with high flexibility can be easily formed.

In addition, the resin layer provided between the tubular structure and the tubular mesh is made of a chemical resistant rubber material or a rubber material having a perfluoro monomer structure, whereby chemical resistance is obtained on the inner side of the outer sheath layer made of a urethane resin or the like, and adaptation to gas sterilization and autoclaving sterilization (by water vapor heated and pressurized to 2 atmospheres and 132° C.) is realized.

### BRIEF DESCRIPTION OF THE DRAWINGS

FIGS. 1A and 1B are explanatory views of the construction of the flexible tube for an endoscope according to the first embodiment of the invention;

FIG. 2 is an explanatory view of the construction of the flexible tube for an endoscope according to the second embodiment of the invention;

FIG. 3 is a molecular structure of the rubber material having the perfluoro monomer structure;

FIGS. 4A and 4B are explanatory views of the construction of the flexible tube for an endoscope according to the third embodiment of the invention;

FIG. 5 is an explanatory view of the entire construction of an endoscope;

FIG. 6 is an explanatory view of the construction of a flexible tube for an endoscope according to the related-art technique.

FIG. 7 is an explanatory view of the entire construction of the endoscope according to the invention;

FIG. 8 is an explanatory view of the construction of the flexible tube for the endoscope;

FIG. 9 is an explanatory view of the construction of the flexible tube for an endoscope according to the related-art technique;

FIGS. 10A and 10B are explanatory views of a construction of a flexible tube for the endoscope according to the invention;

FIGS. 11A and 11B are sectional views of the flexible tubes for the endoscope according to the invention; and

FIGS. 12A to 12E are explanatory views showing a manufacturing example of the flexible tube for the endoscope.

### DETAILED DESCRIPTION OF THE INVENTION

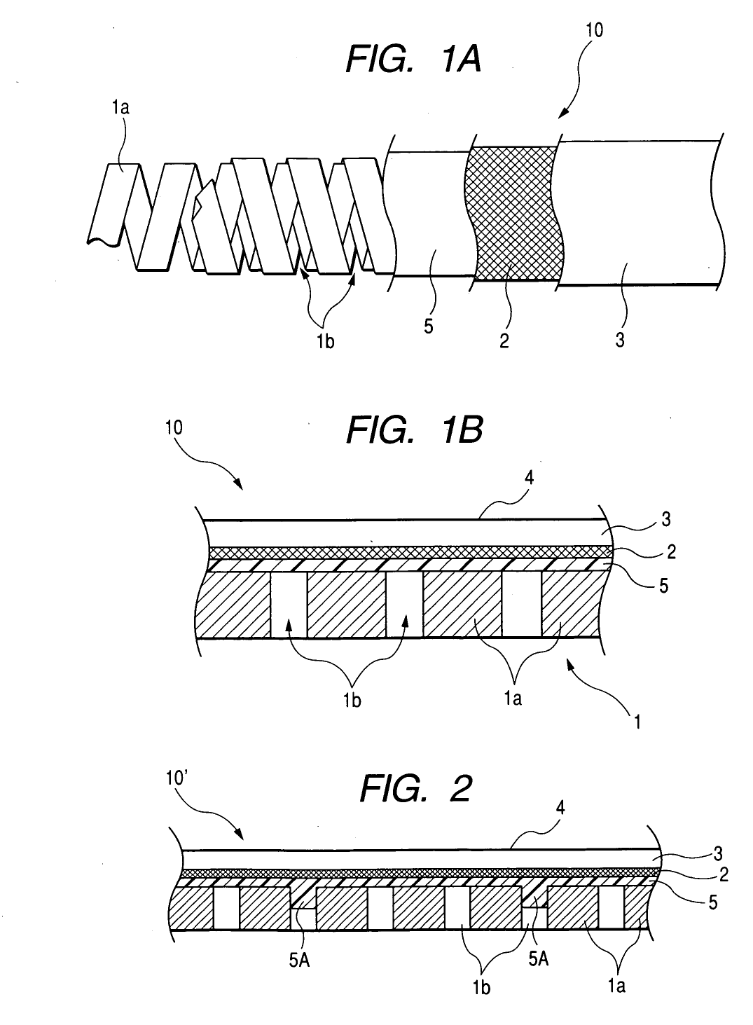

Flexible tubes for an endoscope according to embodiment 1 of the invention are explained with reference to FIGS. 1A and 1B through FIGS. 4A and 4B.

FIGS. 1A and 1B are drawings describing a flexible tube for an endoscope according to an embodiment 1-1 of the invention.

A flexible tube portion occupying the greater part of the length of an insertion portion of an endoscope needs to have flexibility across almost the entire length, and in particular, the portion to be inserted into a body cavity must have a structure with higher flexibility. Herein, the flexibility required as a flexible tube forming the flexible tube portion is flexibility in the bending direction, and a sufficient strength is required in an expanding and contracting direction and a crushing direction.

The flexible tube 10 shown in FIGS. 1A and 1B is formed so that a tubular structure 1 made of a spiral tube formed by spirally winding a metal band 1a at the innermost side is covered by a tubular mesh 2 formed by weaving a metal wire, and on this tubular mesh 2, an outer sheath layer 3 made of a urethane resin or the like is laminated and bonded.

In addition, a coating film 4 containing silicon or the like having chemical resistance is coated on the outer surface of the outer sheath layer 3, and between the tubular structure 1 and the tubular mesh 2, a tube layer 5 made of a rubber material having a perfluoro monomer structure or a chemical resistant rubber material such as fluorine rubber or EPDM (ethylene propylene diene ternary copolymer) is provided, whereby preventing the outer sheath layer 3 made of a urethane resin or the like from being broken (corroded, etc.) during sterilization.

In the flexible tube 10 for an endoscope provided with a tube layer 5 formed from a chemical resistant rubber material between the tubular structure 1 and the tubular mesh 2, the entirety of the outer circumference of the tubular structure 1 is covered by the tube layer 5 made of a chemical resistant rubber material, so that the sterilizing gas entering inside of the endoscope for gas sterilization is stopped by the tube layer 5, and the outer sheath layer 3 is not exposed to the sterilizing gas and there is no possibility that the outer sheath layer 3 made of a urethane resin is broken from the inner side.

In addition, the outer sheath layer 3 made of a urethane resin is bonded and laminated on the outer circumference of the tubular mesh 2, so that the adhesiveness between the tubular mesh 2 and the outer sheath layer 3 is high, and there is no possibility that the outer sheath layer 3 separates from the tubular mesh 2.

Subsequently, a flexible tube 10′ for an endoscope according to an embodiment 1-2 of the invention is explained with reference to FIG. 2.

In the embodiment shown in FIG. 2, when a tube layer 5 made of a chemical resistant rubber material is provided between the tubular structure 1 and the tubular mesh 2, on the outer circumference of the tubular structure 1 made of a spiral tube formed by spirally winding the metal band 1a, a tube layer 5 having convex portions 5A formed on the inner side is provided, and by interposing the convex portions 5A at arbitrary pitches in between-band portions 1b between the metal bands 1a, the hardness of the flexible tube 10′ can be adjusted.

In the section of the flexible tube 10′ shown in FIG. 2, a convex portion 5A formed on the tube layer 5 is interposed every two between-band portions 1b, however, to adjust the tube to be more flexible, the convex portions 5A are disposed on the inner side of the tube layer 5 so that the pitches of the convex portions A to be interposed in the between-band portions 1b are widened (for example, the convex portions are interposed every 5 between-band portions 1b).

Namely, according to the embodiment, the hardness of the flexible tube can be easily adjusted by adjusting the pitches of the convex portions 5A to be interposed in the between-band portions 1b.

In both embodiments 1-1 and 1-2, it is preferable that the tube layer 5 made of a rubber material having a perfluoro monomer structure is provided between the tubular structure 1 and the tubular mesh 2.

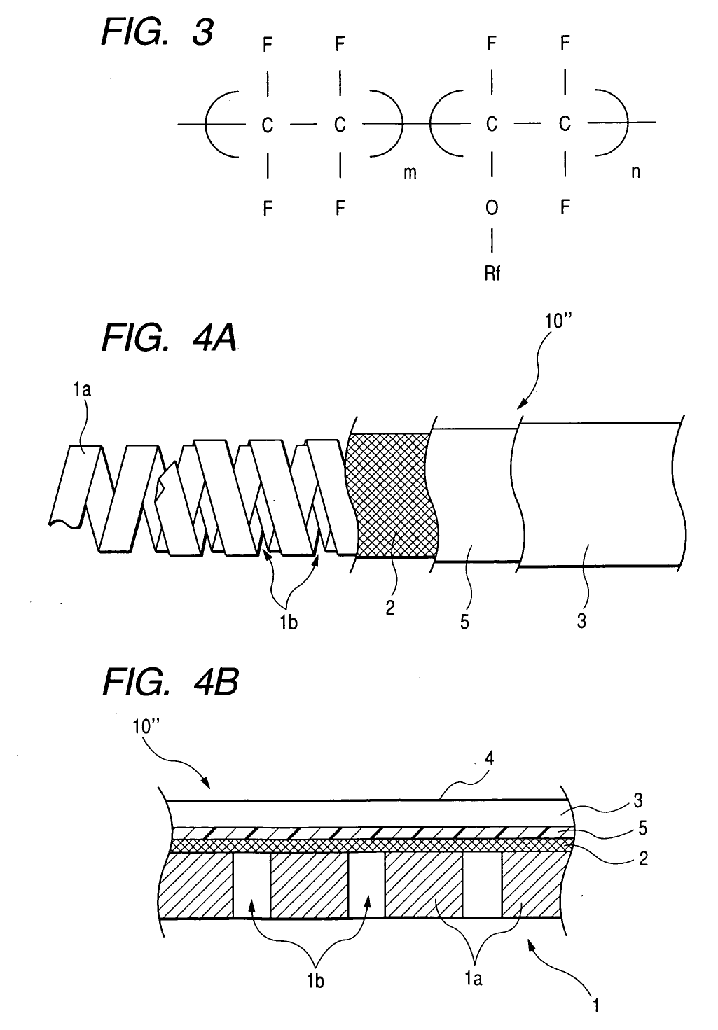

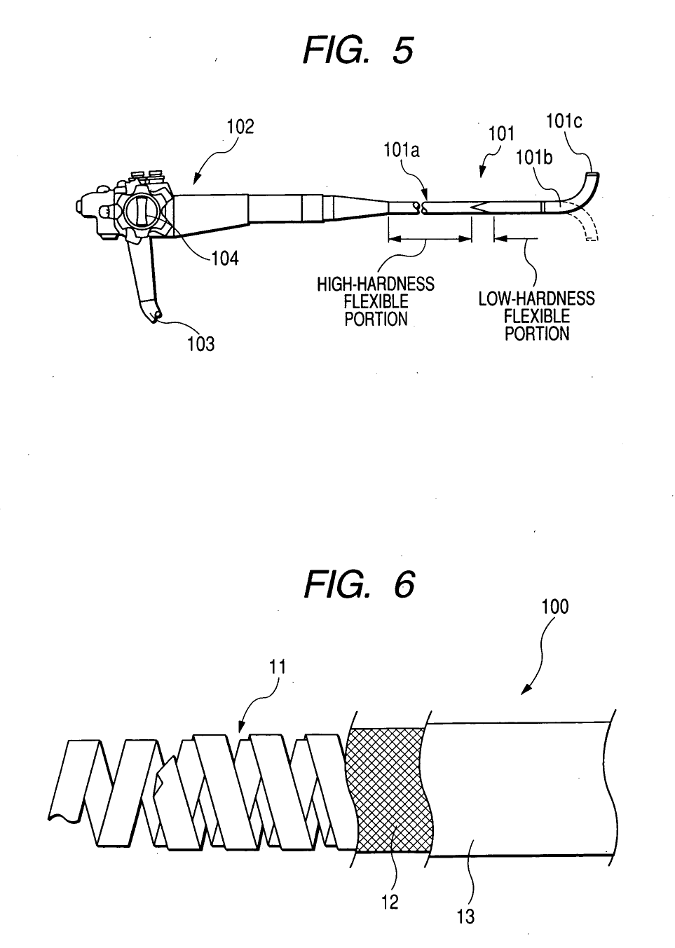

FIG. 3 shows a molecular structure of the rubber material having the perfluoro monomer structure. In this figure, Rf denotes an alkyl group.

The perfluoro monomer structure is composed only by carbon, fluorine, and oxygen, and is similar to the structure of a fluorine resin called PFA (ethylene tetrafluoride-perfluoroalkyl vinylether copolymer). Therefore, its property is similar to that of PFA, and has the following advantages.

(1) The structure is perfectly fluorinated, so that it is excellent in chemical resistance and is not deteriorated even by a new chemical with high oxidizing power.

(2) Heat resistance limit of almost 300° C. (generally 287° C. or less), and adaptable for autoclaving.

(3) The structure has no toxicity, and is available for medical equipment such as endoscopes.

(4) The structure is small in friction resistance and excellent in smoothness, so that there is no possibility that the tube layer is buckled when bending.

(5) Mechanical strength higher than silicon.

Due to these advantages, the high-molecule material with the perfluoro monomer structure can be said to be suitable for medical equipment.

However, the high-molecule material with the perfluoro monomer structure is generally used as a fluorine resin, and its elongation and elasticity as properties unique to rubber are lost, and in the worst cases, the material plastically deforms.

Therefore, the high-molecule material with the perfluoro monomer structure is formed at an average molecular weight of 2000 or less, and this is further vulcanized. The smaller the molecular weight, the more flexible the high-molecule material. Therefore, by setting the average molecular weight to be smaller than that of the resin (with an average molecular weight of 2100 through 9200, normally), the rigidity of the resin is lost and a flexible high-molecule material is obtained.

This high-molecule material is further vulcanized, whereby crosslinking reaction occurs in the high-molecular material, and two-dimensional linear monomer becomes a three-dimensional network, whereby showing a property of elasticity. Thereby, a rubber material excellent in chemical resistance, heat resistance, and mechanical resistance, etc., that is, a rubber material that can adapt to gas sterilization by using a sterilizing gas such as hydrogen peroxide plasma and autoclaving sterilization, and is usable at a sliding portion, can be obtained. The molecular weight and the degree of vulcanization are adjusted so that the molded rubber hardness becomes 60 through 70. As the vulcanization, it is general that a crosslinking agent such as peroxide of 1,1-di(t-butyl peroxy)-3,3,5-trimethyl siloxane or sulfur is mixed and heated, however, other chemical reagents (such as amine, phenol resin), or energy (for example, ultraviolet ray, electron beam, X-ray, etc.) other than heat can also be used.

Next, a flexible tube 10″ for an endoscope according to an embodiment 1-3 of the invention is explained with reference to FIG. 4.

In this embodiment, a tube layer 5 made of a rubber material having the perfluoro monomer structure is provided further inward than the outer sheath layer 3 made of a urethane resin.

As shown in FIG. 4, in the flexible tube 10″ provided with a tube layer 5 made of a rubber material having the perfluoro monomer structure between the outer sheath layer 3 and the tubular mesh 2, a tube layer 5 excellent in chemical resistance (rubber material having the perfluoro monomer structure) covers the entirety of the outer circumference of the tubular mesh 2, so that the sterilizing gas entering inside the endoscope during gas sterilization is stopped by the tube layer 5, so that the outer sheath layer 3 is not exposed to the sterilizing gas from the inner side, and the outer sheath layer 3 made of a urethane resin is not broken from the inner side.

The tube layer 5 made of the rubber material with the perfluoro monomer structure is only required to cover the inner side of the outer sheath layer 3 made of the urethane resin, and the tube layer can be provided at a position with no contact with the outer sheath layer 3 made of a urethane resin.

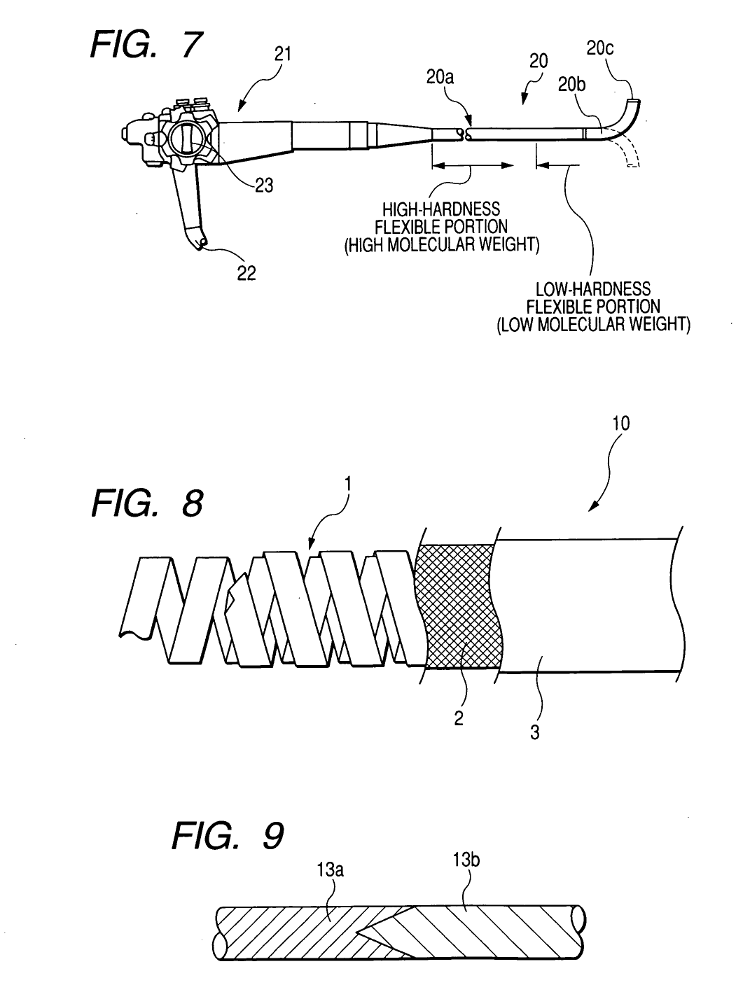

A flexible tube for an endoscope according to an embodiment 2 of the invention is explained with reference to FIGS. 7 and 8.

FIG. 7 is a drawing of the entire construction of the endoscope according to the embodiment of the invention.

As shown in FIG. 7, in the endoscope, the base end of the insertion portion 20 is coupled to the main body control portion 21, and a light guide flexible portion 22 extends from the main body control portion 21. In the insertion portion 20, the greater part of the length from the side coupled to the main body control portion 21 is a flexible tube portion 20a, and at the distal end of the flexible tube portion 20a, an angle portion 20b that can be bent upward, downward, leftward, and rightward by an angle control means 23 provided at the main body control portion 21 is provided, and to the distal end of this angle portion 20b, a distal end main body 20c is coupled. This is not very different from the construction of the related-art technique.

FIG. 8 is a drawing describing the flexible tube 10 forming the flexible tube portion 20a whose degree of flexibility in the bending direction changes in the axial direction.

The flexible tube portion 20a occupying the greater part of the length of the insertion portion of the endoscope needs to have flexibility across almost the entire length, and in particular, the portion to be inserted into a body cavity must have high flexibility. Herein, the flexibility required as a flexible tube forming the flexible tube portion is flexibility in the bending direction, and a sufficient strength is needed in the expanding and contracting direction and the crushing direction.

Therefore, the flexible tube 10 forming the flexible tube portion 20a is formed so that a tubular structure 1 formed of a spiral tube obtained by spirally winding a metal band at the innermost side is covered by a tubular mesh 2 formed by weaving a metal wire, and on the outer circumference of this tubular mesh 2, an outer sheath layer 3 made of a vulcanizing-type rubber material having a perfluoro monomer structure with a molecular weight of 2000 or less is laminated. The vulcanizing-type rubber material having a perfluoro monomer structure and the crosslinking agent are as set forth above.

According to the invention, when the rubber material for forming the outer sheath layer 3 of the flexible tube 10 is crosslinked, the molecular weight of the rubber material is arbitrarily changed in a range between 1000 and 2000 along the axial direction, whereby a hard portion and a flexible portion are formed in the rubber material forming the outer sheath layer 3, and the degree of flexibility of the flexible tube 10 in the bending direction changes in the axial direction.

Namely, in the endoscope shown in FIG. 7, by using the flexible tube 10 in which molecular weight of the rubber material is arbitrarily changed in a range between 1000 and 2000 along the axial direction and the rubber material is crosslinked for forming the outer sheath layer 3, the degree of flexibility of the flexible tube portion 20a in the bending direction changes in the axial direction.

In this embodiment, the crosslinking reaction is controlled by changing the molecular weight of the rubber material so that a predetermined length (base end side) of the flexible tube portion 20a from the side coupled to the main body control portion 21 is formed into a high-hardness flexible portion in which the molecular weight of the rubber material forming the outer sheath layer 3 is comparatively high, and a predetermined length (angle side) from the side coupled to the angle portion 20b is formed into a low-hardness flexible portion in which the molecular weight of the rubber material forming the outer sheath layer 3 is comparatively low.

Herein, the high-hardness flexible portion is a portion that has high resistance against bending although keeping flexibility in the bending direction, that is, a harder portion, and the low-hardness flexible portion is a portion with a resistance against bending smaller than that of the high-hardness flexible portion, that is, a more flexible portion. Thus, there is a hardness difference in the bending direction between the high-hardness flexible portion and the low-hardness flexible portion, and the difference is properly set based on the insertion resistance, the degree of curve of the insertion path, and the purpose of use.

Incidentally, the crosslinking reaction (vulcanization) can be controlled by changing the amount of peroxide, which is a crosslinking agent, the amount of irradiation of ultraviolet rays, electron beams, or X-rays.

When the vulcanizing-type rubber material having the perfluoro monomer structure with a molecular weight of 2000 or less is crosslinked (vulcanized), the lower the molecular weight of the rubber material, the more flexible the rubber material, however, if the molecular weight is less than 1000, the resistance against a disinfectant such as hydrogen peroxide plasma (chemical resistance) and durability for autoclaving sterilization become low, so that it is preferable that the molecular weight is arbitrarily changed in a range between 1000 and 2000.

A flexible tube for an endoscope according to an embodiment 3 of the invention is explained with reference to FIGS. 7, 10A, 10B, 11A, 11B, and 12A to 12E.

FIG. 7 shows the entire construction of the endoscope according to the embodiment 3 of the invention.

As shown in FIG. 7, in the endoscope, the base end of the insertion portion 20 is coupled to the main body control portion 21, and a light guide flexible portion 22 extends from the main body control portion 21. In the insertion portion 20, the greater part of the length from the coupled side to the main body control portion 21 is a flexible tube portion 20a, and to the distal end of the flexible tube portion 20a, an angle portion 20b that can be bent upward, downward, leftward, and rightward by an angle control means 23 provided at the main body control portion 21 is coupled, and to the distal end of this angle portion 20b, a distal end main body 20c is coupled. This is not very different from the construction of the related-art technique.

The flexible tube portion 20a occupying the greater part of the length of the insertion portion 20 of the endoscope needs to have flexibility across almost the entire length, and in particular, the portion to be inserted into a body cavity must have higher flexibility. Herein, the flexibility required as a flexible tube 10 forming the flexible tube portion 20a is flexibility in the bending direction, and a sufficient strength is needed in the expanding and contracting direction and the crushing direction.

In addition, in order to obtain an excellent pushing thrust to insert the flexible tube portion 20a into a body cavity, desirably, the base end side of the flexible portion 20a has considerably high rigidity against bending, and on the other hand, desirably, the angle side of the flexible tube portion 20a has a higher degree of flexibility so that, when the angle portion 20b bends, it follows the bend of the angle portion 20b to some degree and smoothly bends along the curved insertion path. Therefore, it is necessary in terms of inserting operability and pain reduction for a patient that the degree of flexibility of the flexible tube portion 101b is changed in the axial direction.

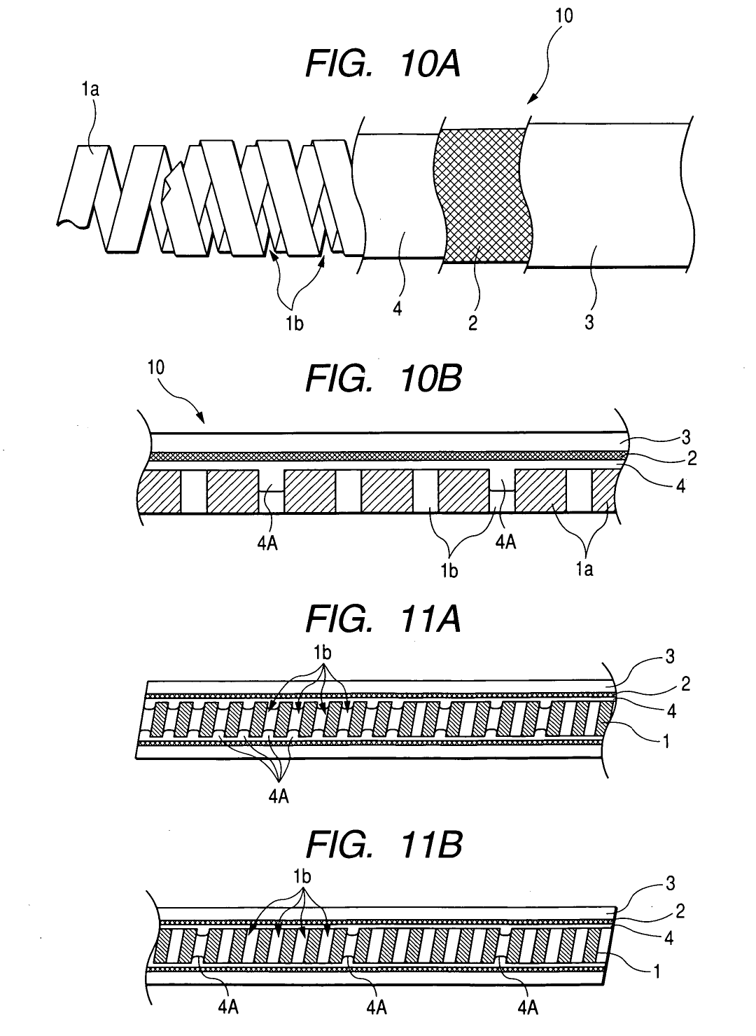

FIGS. 10A and 10B are drawings describing the flexible tube 10 for forming a flexible tube portion 20a which has flexibility in its bending direction and has a sufficient strength in its expanding and contracting direction and crushing direction, and changes the degree of flexibility in the bending direction in the axial direction.

According to the invention, as shown in FIG. 10A, the tubular structure 1 formed of a spiral tube formed by spirally winding a metal band at the innermost side is covered by a tubular mesh 2 formed by weaving a metal wire, and on the outer circumference of the tubular mesh 2, an outer sheath layer 3 made of a urethane resin or the like is covered, whereby a flexible tube 10 for an endoscope having flexibility across almost the entire length in its bending direction is constructed.

Thereby, a flexible tube 10 that has flexibility in its bending direction and a sufficient strength in its expanding and contracting direction and crushing direction can be obtained.

In addition, between the tubular structure 1 and the tubular mesh 2, a thin-film resin layer 4 having convex portions 4A formed on the inner side is provided, and as shown in the partial sectional view of FIG. 10B, the plurality of convex portions 4A are formed on the inner side of the resin layer 4 and are interposed in between-band portions 1b of the spiral tube at arbitrary pitches.

By arbitrarily changing the pitches of the convex portions 4A of the resin layer 4 provided between the tubular structure 1 and the tubular mesh 2, interposed in between-band portions 1b of the spiral tube, the degree of flexibility in the bending direction of the flexible tube 10 is changed in the axial direction.

In the flexible tube portion 10 of this embodiment, the convex portions 4A formed on the inner side of the thin-film resin layer 4 provided between the tubular structure 1 and the tubular mesh 2 are interposed in between-band portions 1b of the spiral tube at arbitrary pitches, and by interposing the convex portions 4A in the between-band portions 1b of the spiral tube at narrow pitches, a high-hardness flexible portion is formed, and by interposing the convex portions 4a at wide pitches, a low-hardness flexible portion is formed.

Herein, the high-hardness flexible portion is high in resistance against bending, that is, hard although it has flexibility in its bending direction, and the low-hardness flexible portion is smaller in resistance against bending, that is, more flexible than the high-hardness flexible portion. Thus, there is a hardness difference in the bending direction between the high-hardness flexible portion and the low-hardness flexible portion, and the difference is properly set depending on the insertion resistance, the degree of curve of the insertion path, and the purpose of use.

The flexible tube 10 whose degree of flexibility in the bending direction is changed in the axial direction is explained with reference to FIGS. 11A and 11B.

FIG. 11A is a sectional view of the flexible tube at a portion where the pitches of the convex portions interposed in the between-band portions of the spiral tube are narrowed, and FIG. 11B is a sectional view of the flexible tube at a portion where the pitches of the convex portions interposed in the between-band portions of the spiral tube are widened.

For example, the portion of the base end side of the flexible tube portion 20a can be formed into a high-hardness flexible portion by interposing the convex portions 4A of the resin layer 4 provided between the tubular structure 1 and the tubular mesh 2, in the between-band portions 1b of the spiral tube at narrow pitches as shown in FIG. 11A.

On the other hand, for example, the portion of the angle side of the flexible tube portion 20a can be formed into a low-hardness flexible portion by interposing the convex portions 4A of the resin layer 4 provided between the tubular structure 1 and the tubular mesh 2, in the between-band portions 1b of the spiral tube at wide pitches as shown in FIG. 11B.

Namely, in the flexible tube 10 of FIGS. 11A and 11B, the resin layer 4 is provided between the tubular structure 1 and the tubular mesh 2, the convex portions 4A on the inner side of the resin layer 4 are interposed in the between-band portions 1b of the spiral tube at arbitrary pitches, and by controlling the pitches, the degree of flexibility in the bending direction is changed in the axial direction.

In addition, it is preferable that a resin layer 4 made of a chemical resistant resin material is provided between the tubular structure 1 and the tubular mesh 2.

When the outer sheath layer 3 formed on the outer circumference of the tubular mesh 2 is made of a urethane resin, the urethane resin is low in durability against chemicals for cleaning and sterilizing the endoscope, so that a chemical resistant coating film is formed on the surface of the outer sheath layer 3 made of the urethane resin, and the resin layer 4 made of a chemical resistant resin material is provided between the tubular structure 1 and the tubular mesh 2, whereby the outer sheath layer 3 can be protected from the outer side and the inner side.

It is more preferable that a resin layer 4 made of a rubber material having a perfluoro monomer structure explained in the above-mentioned embodiment 1 is provided.

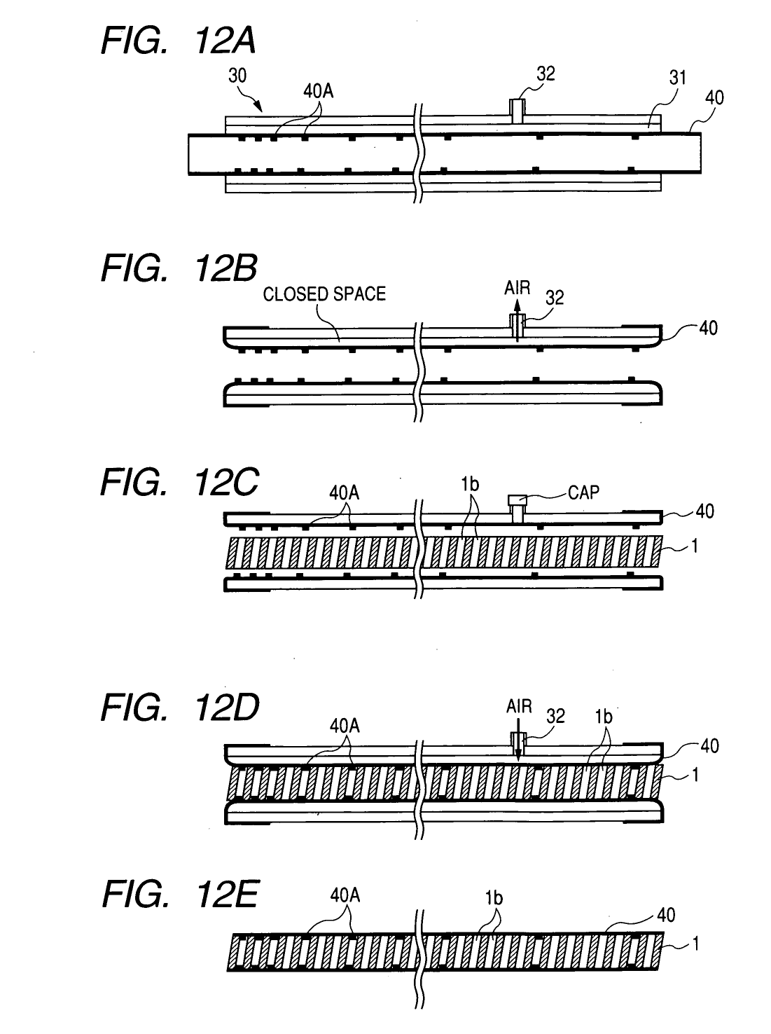

Next, with reference to FIGS. 12A to 12E, a method for manufacturing the flexible tube 10 in which a thin-film resin layer 4 having convex portions 4A formed on the inner side is provided between the tubular structure 1 and the tubular mesh 2, and the degree of flexibility in the bending direction is changed in the axial direction, is explained.

In the embodiment shown in FIGS. 12A to 12E, a tube member 40 having convex portions 40A on the inner side is covered on the outer circumference of the tubular structure 1 formed of a spiral tube, and then the tubular mesh 2 and the outer sheath layer 3 are provided on the outer circumference of the tube member, whereby the resin 1 is provided between the tubular structure 1 and the tubular mesh 2, and the convex portions 4A formed on the inner side of the resin layer 4 are interposed in between-band portions 1b of the spiral tube at arbitrary pitches.

Herein, in the embodiment shown in FIGS. 12A to 12E, by using a circular tube-shaped jig 30, the thin-film tube member 40 made of a resin material is covered on the outer circumference of the tubular structure 1 formed of a spiral tube.

The circular tube-shaped jig 30 has a through hole 31 with a sufficient opening for penetrating the tubular structure 1 formed of a spiral tube.

The tube member 40 to be covered on the outer circumference of the tubular structure 1 formed of a spiral tube is formed so as to match its inner diameter with the outer diameter of the tubular structure 1, and on the inner side of the tube member 40, a plurality of convex portions 40A are formed. The convex portions 40A formed on the inner side of the tube member 40 are formed at arbitrary pitches corresponding to the positions of the between-band portions 1b of the spiral tube.

As shown in FIG. 12A, through the through hole 31 of the circular tube-shaped jig 30, the thin-film tube member 40 made of a resin material is penetrated, and as shown in FIG. 12B, left and right ends of the tube member 40 are folded outward from the opening to form a closed space between the jig 30 and the tube member 40.

Next, the air inside the closed space is suctioned from a vent hole 32 of the circular tube-shaped jig 30, and as shown in FIG. 12C, the tube member 40 is deformed along the inner wall of the circular tube-shaped jig 30, and then the tubular structure 1 is penetrated through the inside of the tube member 40 whose tube diameter has been expanded.

In this embodiment, after the air in the closed space is suctioned through the vent hole 32, the vent hole 32 is closed by a cap or the like, whereby a vacuum state is obtained between the jig 30 and the tube member 40, and due to the air pressure difference, the tube member 40 is deformed. Then, on the inner side of the tube member 40 whose tube diameter has been expanded, the tubular structure 1 formed of a spiral tube is disposed.

Thereafter, as shown in FIG. 12D, the vent hole 32 is opened to supply air between the jig 30 and the tube member 40, the tube member 40 with an expanded tube diameter is restored to the original state, and the tube member 40 is covered on the outer circumference of the tubular structure 1 formed of a spiral tube so that the convex portions 40A formed on the inner side of the tube member 40 are interposed in the between-band portions 1b of the spiral tube.

Then, as shown in FIG. 12E, the tube member 40 is removed from the jig 30, and from the through hole 31 of the jig 30, the tubular structure 1 whose outer circumference is covered by the tube material 40 is extracted.

The tubular mesh 2 formed by weaving a metal wire is covered on the outer circumference of the tubular structure 1 covered by the tube member 40, and the outer sheath layer 3 made of a urethane resin or the like is laminated on the tubular mesh 2, whereby the flexible tube 10 for an endoscope is formed in which a resin layer 4 is provided between the tubular structure 1 and the tubular mesh 2 and the convex portions 4A formed on the inner side of the resin layer 4 are interposed at arbitrary pitches in between-band portions 1b of the spiral tube.

Namely, in the embodiment shown in FIGS. 12A to 12E, before covering the tube member 40 made of a resin material on the tubular structure 1 formed of a spiral tube, for a portion where a high-hardness flexible portion is desired to be formed, convex portions 40A are formed in advance at narrow pitches on the inner side of the tube member 40, and for a portion where a low-hardness flexible portion is desired to be formed, convex portions 40A are formed in advance at wide pitches on the inner side of the tube member 40.

Then, when the tube member 40 is covered on the tubular structure 1 formed of a spiral tube, the convex portions 40A formed on the inner side of the tube member 40 are interposed in the between-band portions 1b of the spiral tube, whereby the flexible tube 10 for the endoscope whose degree of flexibility in the bending direction is changed in the axial direction is formed.

The entire disclosure of each and every foreign patent application from which the benefit of foreign priority has been claimed in the present application is incorporated herein by reference, as if fully set forth.

What is claimed is:

1. A flexible tube which is for an endoscope and has flexibility across almost an entire length in its bending direction, the flexible tube comprising:
a tubular structure with flexibility in its bending direction;
a tubular mesh that covers the tubular structure; and
an outer sheath layer comprising a urethane resin, the outer sheath layer being laminated on the tubular mesh,
wherein a tube layer formed from a chemical resistant rubber material is provided between the tubular structure and the tubular mesh.

2. The flexible tube for an endoscope according to claim 1,
wherein the tubular structure is a spiral tube formed by spirally winding a metal band, the spiral tube comprising between-band portions,
the tube layer comprises convex portions formed on its inner side and is provided on an outer circumference of the tubular structure, and
the convex portions are interposed at arbitrary pitches in the between-band portions of the spiral tube.

3. The flexible tube for an endoscope according to claim 1,
wherein the chemical resistant rubber material is a rubber material having a perfluoro monomer structure.

4. A flexible tube which is for an endoscope and has flexibility across almost an entire length in its bending direction, the flexible tube comprising:
a tubular structure with flexibility in a bending direction;
a tubular mesh that covers the tubular structure; and
an outer sheath layer comprising a urethane resin laminated on the tubular mesh,
wherein a tube layer formed from a rubber material having a perfluoro monomer structure is provided further inward than the outer sheath layer.

5. A flexible tube which is for an endoscope and has flexibility across almost an entire length in its bending direction, the flexible tube comprising:
a tubular structure with flexibility in its bending direction;
a tubular mesh that covers the tubular structure; and
an outer sheath layer laminated on this tubular mesh,
wherein the outer sheath layer is formed by crosslinking a vulcanizing-type rubber material having a perfluoro monomer structure, and
a molecular weight of the rubber material is arbitrarily changed in a range between 1000 and 2000 along an axial direction of the outer sheath layer, so that degree of flexibility in the bending direction is changed in the axial direction.

6. The flexible tube for an endoscope according to claim 5,
wherein the vulcanizing-type rubber material is vulcanized by using a crosslinking agent so as to form the outer sheath layer.

7. The flexible tube for an endoscope according to claim 5,
wherein the molecular weight of the rubber material in the outer sheath layer on a base end side of the flexible tube is higher to form a high-hardness flexible portion with low flexibility, and the molecular weight of the rubber material in the outer sheath layer on an angle side of the flexible tube is lower to form a low-hardness flexible portion with high flexibility.

8. A flexible tube which is for an endoscope and has flexibility across almost an entire length in its bending direction, the flexible tube comprising:
a tubular structure with flexibility in its bending direction;
a tubular mesh that covers the tubular structure;
an outer sheath layer comprising a rubber material, the outer sheath layer laminated on the tubular mesh,
wherein the tubular structure is a spiral tube formed by spirally winding a metal band, the spiral tube comprising between-band portions,
a thin-film resin layer having convex portions formed on its inner side is provided between the tubular structure and the tubular mesh so that the convex portions of the resin layer are interposed at arbitrary pitches in between-band portions of the spiral tube, and
degree of flexibility in the bending direction is changed in the axial direction by arbitrarily changing the pitches of the convex portions interposed in the between-band portions of the spiral tube along an axial direction of the spiral tube.

9. The flexible tube for an endoscope according to claim 8, comprising:
a high-hardness flexible portion formed by interposing the convex portions in the between-band portions of the spiral tube at narrower pitches; and
a low-hardness flexible portion formed by interposing the convex portions in the between-band portions of the spiral tube at wider pitches.

10. The flexible tube for an endoscope according to claim 8,
wherein the thin-film resin layer is formed from a chemical resistant rubber material.

11. The flexible tube for an endoscope according to claim 8,
wherein the thin-film resin layer is formed from a rubber material having a perfluoro monomer structure.
